(19) United States
(12) Plant Patent Application Publication
(10) Pub. No.: US 2021/0176908 P1
(43) Pub. Date: Jun. 10, 2021

(54) RASPBERRY PLANT NAMED 'HALLEY'

(50) ;

(71) Applicants: Aldo Telch, Faver (IT); MOLARI SOCIETA AGRICOLA S.S., Cesena (IT)

(72) Inventor: Aldo TELCH, Faver (IT)

(21) Appl. No.: 17/112,538

(22) Filed: Dec. 4, 2020

(30) Foreign Application Priority Data
Dec. 4, 2019 (QZ) 20193309

(51) Int. Cl. A01H 6/74

(52) U.S. Cl. PLT/204

(57) ABSTRACT

A new very versatile primocane and floricane raspberry cultivar named ‘Halley’ which produces berries which are of a large size with a good colour and low acid aromatic flavour is disclosed. The new cultivar has the advantages of growing medium length, thick completely thornless canes, producing two crops per year, wherein the fruits ripen on primocane in spring and on floricane in summer/autumn.

Genus and species: Rubus idaeus L.

Variety denomination: ‘Halley’.

### BACKGROUND OF THE NEW VARIETY

A new and distinct cultivar of red raspberry (Rubus idaeus L.) hereinafter referred to by the cultivar denomination ‘Halley’ is disclosed. The new variety ‘Halley’ was obtained from a controlled cross between the varieties ‘T51L6 ’ (mother plant) and ‘TD73’ (father plant).

The crossing for the new cultivar occurred in the year 2012 in Altopiano della Vigolana (Trento, Italy) and it was subjected to selection in 2014, and successively tested until 2019. The characteristics of the new cultivar have been found stable and have been transmitted without change through succeeding (more than 7 years) asexual vegetative propagations by root cutting.

Plant breeder's rights were applied for with the European Union on Dec. 4, 2019. ‘Halley’ has not been made publicly available.

### SUMMARY OF THE INVENTION

The most outstanding and distinguishing characteristics of the cultivar ‘Halley’ are hereunder reported.
Early ripening primocane and early ripening floricane raspberry plant variety
Very versatile variety that can adapt to various low chill climates
Medium length thick canes that are completely thornless
Auto-fertile variety
Very vigorous and healthy plant
High yield plant
Low acid, very aromatic berries
Large, conical shaped berries
Light red coloured fruit at harvest

The new variety produces two crops per year and the fruits mainly ripen on primocane in June and on floricane from the beginning of August to the beginning of October.

The new variety can be propagated by: the use of suckers produced in a spawn-bed, root cutting, and tissue culture techniques. Propagation by root cutting is preferred.

### BRIEF DESCRIPTION OF THE PHOTOGRAPHS

The new red raspberry cultivar ‘Halley’ is illustrated by the accompanying photographs showing typical specimens of the new variety in colour as true as reasonably possible to make the same colour illustrations of this character by conventional photographic procedures. The photographs are of plants that are from four to six months old.

FIG. 1 is a view showing the raspberry plant with growing fruits. The upright and tall plants with very vigorous lateral shoots, mainly present on the apical part of the plant, can be appreciated.

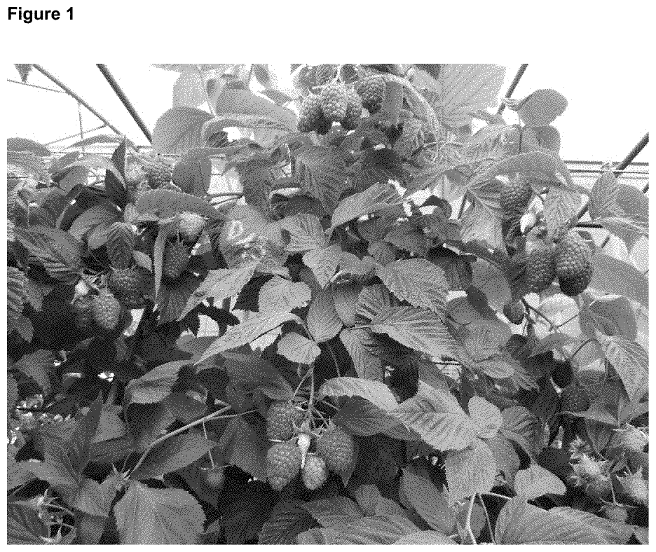

FIG. 2 is a view showing the ripe fruit size and shape.

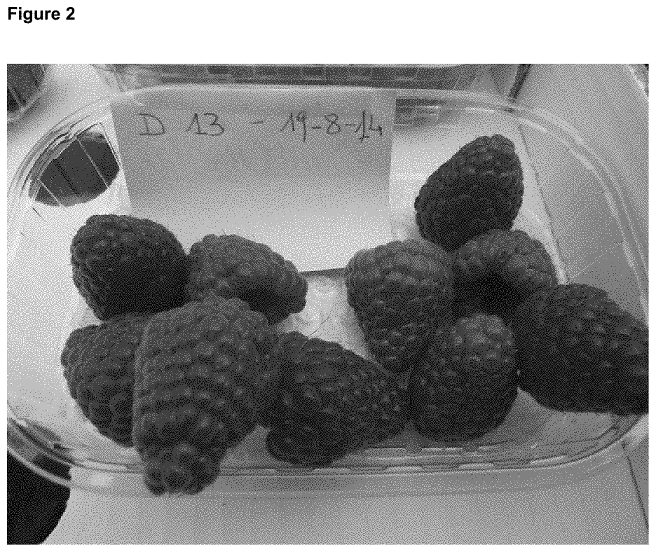

### DETAILED DESCRIPTION

The following detailed description set forth the distinctive characteristics of the new variety ‘Halley’. The performance of the new cultivar for retention of its distinctive characteristics has been evaluated through successive asexual propagations by root cutting in the Applicant's setting at Altopiano della Vigolana, Trentino Region (Italy) under natural conditions in plastic covered high tunnel. The growing was in soilless conditions using coir as substrate.
Classification:
   Family.—Rosaceae.
   Botanical.—Rubus idaeus L.
   Common name.—Raspberry.
Parentage: ‘T51L6’×‘TD73’.
Seasonality of the new ‘Halley’ variety: Early ripening primocane variety, 5 days later than Enrosadira® variety. Early ripening floricane variety, same as Enrosadira® variety.
Climatic areas: Very versatile variety, adapted from Continental to Low Chill Climates, showing its best potential in areas with at least 400 chill hours.
Plant structure and agronomic description: Medium length canes, thick canes, completely thornless. Medium length lateral shoots. Good flower pollination. Auto-fertile variety, but the presence of pollinator insects is always helpful.
Yield: High yield variety, suitable for high density cultivation systems. Really easy to pick and to cultivate, being completely thornless.
Berry description:
   Shape.—Conical shape.
   Size.—medium to large size.
   Firmness.—very firm berries.
   Colour.—light red colour.
   Taste.—sweet, low acid, and very aromatic berries.
   Main bearing type.—only on current year's cane in autumn.
Resistance to pests and diseases: ‘Halley’ is tolerant to mildew, black root, and Phytophtorta r.

TABLE 2
Other comparisons with commercial cultivars

| | | | BERRY | BERRY |
|---|---|---|---|---|
| | | CHILL | WEIGHT | COLOUR |
| VARIETY | SEASONALITY | REQUIREMENT | (g) | (at harvest day) |
| ENROSADIRA ® | Early | Mid-High | 6 | Light red |
| HALLEY | Early | Mid-High | 5, 3 | Red |
| CASTION ® | Early-Mid | Mid-High | 6, 6 | Shiny red |
| DOROTEA | Mid-Late | Mid-High | 7, 1 | Red |
| AURORA ® | Late | Mid-High | 5, 7 | Pink red |
| POLKA | Early | High | 6, 6 | Red |
| AUTUMN BLISS | Early | High | 4 | Red |

TABLE 3
Comparison with Enrosadira ®

| | Plant growth | Presence of Spines |
|---|---|---|
| ENROSADIRA ® | Early | Yes |
| HALLEY | Earlier | No |

The plant assessment was performed for 2 years.

What is claimed:

1. A new and distinct cultivar of raspberry plant as substantially herein shown and described.
